(19) United States
(12) Patent Application Publication
(10) Pub. No.: US 2013/0324508 A1
(43) Pub. Date: Dec. 5, 2013

(54) BIOMARKER FOR PROSTATE CANCER AND METHOD OF USING THE SAME

(71) Applicant: UNIVERSITY OF TENNESSEE RESEARCH FOUNDATION, Knoxville, TN (US)

(72) Inventors: Leigh Darryl Quarles, Memphis, TN (US); Min Pi, Germantown, TN (US)

(73) Assignee: UNIVERSITY OF TENNESSEE RESEARCH FOUNDATION, Knoxville, TN (US)

(21) Appl. No.: 13/908,822

(22) Filed: Jun. 3, 2013

Related U.S. Application Data
(60) Provisional application No. 61/654,563, filed on Jun. 1, 2012.

(51) Int. Cl. C12Q 1/68; G01N 33/74

(52) U.S. Cl. CPC C12Q 1/6886; G01N 33/743
USPC 514/173; 435/6.12; 435/6.11; 435/7.23; 530/389.7; 536/24.33; 536/24.31; 514/182; 514/178; 514/175; 514/618; 514/629; 514/284

(57) ABSTRACT

The present invention relates to a biomarker for characterizing prostate cancer and method of using the same. More particularly, the invention relates to method of using a membrane-associated C family G protein-coupled receptor GPRC6A as biomarker of characterizing prostate cancer progression. The present invention also provides a kit for detecting prostate cancer in a subject.

### RELATED APPLICATION

This application claims priority from U.S. Provisional Application Ser. No. 61/654,563, filed Jun. 1, 2012, the entire disclosure of which is incorporated herein by this reference.

### GOVERNMENT INTEREST

This invention was made with government support under grant number R01-AR37308 awarded by the National Institutes of Health. The government has certain rights in the invention.

### FIELD OF THE INVENTION

The present invention relates to a biomarker for prostate cancer and method of using the same. More particularly, the invention relates to method of using a membrane-associated C family G protein-coupled receptor GPRC6A as biomarker to characterizing prostate cancer progression. The present invention further provides a kit for detecting prostate cancer in a subject.

### BACKGROUND OF THE INVENTION

Prostate cancer is the most commonly diagnosed cancer in men and the second leading cause of death from cancer in North American and European males. New therapeutic approaches are needed to prevent and treat advanced and metastatic prostate cancer. Nutritional factors, particularly high intake of protein and calcium, as well as metabolic syndrome, are known to modify prostate cancer risk and progression, but the molecular mechanisms linking nutrition to prostate cancer are unknown. There are also links between prostate cancer and bone metabolism. Osteocalcin (OC), which encodes a vitamin-K dependent hormone predominantly produced by osteoblasts/osteocytes in bone, which functions to regulate energy metabolism, is also ectopically expressed by some prostate cancers that have a propensity to metastasize to bone. Polymorphisms in OC are also associated with prostate cancer progression. Recent evidence has also identified a correlation between the bone transcription factor Runx2 and advanced stages of prostate and breast cancer, as evidenced by the effects of depletion of Runx2 by RNA interference to inhibit migration and invasive properties of the cells and prevent metastatic bone disease. It is possible that OC secreted by bone may directly target prostate cancer cells. Finally, androgen deprivation therapy is the principal medical therapy for prostate cancer, but androgen ablation often becomes ineffective in controlling prostate cancer progression and castration-resistant metastatic disease, particularly to bone, becomes incurable. There is growing evidence for the presence of a putative membrane androgen sensing receptor that mediates the rapid, non-genomic effects of androgens, which also might be involved in prostate cancer growth and metastasis. Regardless, clues to possible new molecular targets to regulate prostate cancer growth and progression may be discovered from a better understanding of the molecular mechanisms underlying nutritional risk factors, OC effects and androgen resistance in prostate cancer.

GPRC6A, a recently discovered member of family C G protein-coupled receptors, may provide a molecular mechanism to explain the link between prostate cancer progression and nutrition, OC responsiveness of prostate cancer cells, and continued androgen responsiveness in prostate cancer cells following inhibition of nuclear androgen receptor signaling. Recent work has shown that GPRC6A is capable of sensing extracellular calcium as well as amino acids and OC and to regulate a wide range of metabolic processes, suggesting that the physiological function of this G-protein coupled receptor is to link nutrient and OC signals to the regulation of energy metabolism. In addition, ablation of this orphan G-protein coupled receptor leads to undermasculinization associated with decreased muscle mass, increased adiposity, and low circulating testosterone and elevated estradiol levels in male mice, suggesting that GPRC6A may also modulate sex steroid end organ responses. GPRC6A also mediates the non-genomic effects of testosterone in vitro and in vivo. Finally, GPRC6A is one of five novel genetic loci associated with prostate cancer in the Japanese population. Thus, GPRC6A is a candidate for the putative membrane androgen sensing receptor involved in prostate cancer progression as well as a nutrient and OC receptor regulation of prostate cancer growth and progression.

Therefore, it is desirable to develop a new biomarker based on GPRC6A and methods of using same for diagnosing and treating prostate cancer.

### SUMMARY OF THE INVENTION

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In one embodiment, this invention provides a method of characterizing a disease, including but not limited to, prostate cancer, including treatment resistant prostate cancer. This method includes the steps of: determining the level of GPRC6A in a biological sample from the subject; and comparing the level of GPRC6A in the biological sample to a reference, wherein the disease is characterized based on a measurable difference in the level of GPRC6A in the biological sample as compared to the reference. Further, the method involves the step of determining the amount of cyclic AMP in the biological sample of the subject, including determining the amount of cyclic AMP in the biological sample from the subject and comparing the amount of cyclic AMP in the biological sample to a reference, wherein the disease is characterized based on a measurable difference in the amount of cyclic AMP in the biological sample as compared to the reference.

In another embodiment, the present invention provides for the isolation of GPRC6A gene or gene products by performing an in vitro assay to determine the expression levels of GPRC6A gene or gene products. Further, the in vitro assay can include immunoassay, histological or cytological assay, quantitative real-time PCR, and mRNA expression level assay.

In another embodiment, the present invention provides a kit for detecting prostate cancer in a subject, the kit comprising detecting GPRC6A in a biological sample of the subject. The kit can contain probes, primers and antibodies for detecting GPRC6A in the sample. The increase in the level of GPRC6A can be used to diagnose prostate cancer and is a prognostic indicator that the subject relative to a predetermined level of GPRC6A in the sample, has an increased likelihood of aggressive prostate cancer cells, and prostate cancer growth, malignancy, and poor survival.

In another embodiment, the present invention provides a method of modulating prostate cancer progression and treating treatment-resistant prostate cancer. This method includes the steps of administering to a subject with prostate cancer a therapeutically effective amount of an androgenergic antagonist.

In yet another embodiment of the present invention, the likelihood of a positive therapeutic effect of the androgenergic antagonist can be predicted by determining the amount of cyclic AMP before and after administration of the androgenergic antagonist.

In one embodiment of the presnet invention, the antagonist can be allylestrenol, oxendolone, osaterone acetate, bicalutamide, steroidal anti-androgergic agents, medroxyprogesterone (MPA), cyproterone, cyproterone acetate (CPA), dienogest, flutamide, nilutamide, spironolactone, 5alpha-reductase inhibitors, dutasteride, finasteride, salts thereof, gold nanoparticles thereof, combinations thereof, and the like.

In a preferred embodiment of the present invention, the antagonist is bicalutamide, α-bicalutamide, and β-bicalutamide, and gold nanoparticles thereof.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE SEVERAL VIEWS OF THE DRAWINGS

For a fuller understanding of the nature and desired objects of the present invention, reference is made to the following detailed description taken in conjunction with the accompanying drawing figures.

FIG. 1 illustrates that the expression of GPRC6A in normal human prostate gland (A), the over-expression of GPRC6A in human prostate cancer cell lines and human prostate cancer tissue (B) and (C), cAMP production in response to GPRC6A stimulation (D).

FIGS. 2 (A) and (B) show that the ligands of GPRC6A stimulated ERK activation in human prostate cancer cell lines.

FIG. 3 shows that the ligands, Calcium (A) and Osteocalcin (B) of GPRC6A stimulated human prostate cancer cell proliferation and gene expression.

FIG. 4 shows the ligands of GPRC6A stimulated human prostate cancer cells gene expression. OC, arginine and R1881 stimulated PSA and Runx 2II gene expression in human prostate cancer 22Rv1 (A and B) and PC-3 cells (C and D).

FIG. 5 shows GPRC6A siRNAs inhibited GPRC6A-mediated activation of phosph-ERK in human prostate cancer cell lines. A). GPRC6A siRNAs, hGPRC6A siRNA-202 and siRNA-514 inhibited GPRC6A mRNA expression in 22Rv1 and PC-3 cells. B) GPRC6A-mediated OC and testosterone stimulated phospho-ERK activation blocked by transfecting hGPRC6A siRNA-202 and siRNA-514 in 22Rv1 and PC-3 cells. C). GPRC6A-mediated calcium, testosterone, arginine and OC stimulated phospho-ERK activation blocked by hGPRC6A siRNA-202 in 22Rv1 cells. Representative blots are shown, and the results were verified in at least three independent experiments.

FIG. 6 shows that GPRC6A siRNAs inhibited GPRC6A-mediated stimulation gene expression of PSA (A) and Runx2 (B) and activation of cell chemotaxis in human prostate cancer cell lines (C).

FIG. 7 illustrates the effects of superimposed Gprc6a deficiency in the TRAMP mouse. (A) shows the gross appearance of whole prostatic glands (Upper panel) and hematoxylin/eosin stained histological sections of ventral prostate from Gprc6a^{-/-}, TRAMP and Gprc6a^{-/-}/TRAMP mice, and (B) shows comparison of the survival rates in TRAMP and compound Gprc6a^{-/-}/TRAMP mice.

### DETAILED DESCRIPTION OF THE INVENTION

The details of one or more embodiments of the presently disclosed subject matter are set forth in the accompanying description below. Other features, objects, and advantages of the presently disclosed subject matter will be apparent from the detailed description, Appendix, and claims. All publications, patent applications, patents, and other references mentioned herein are incorporated by reference in their entirety. Some of the polynucleotide and polypeptide sequences disclosed herein are cross-referenced to GENBANK® accession numbers. The sequences cross-referenced in the GENBANK® database are expressly incorporated by reference as are equivalent and related sequences present in GENBANK® or other public databases. Also expressly incorporated herein by reference are all annotations present in the GENBANK® database associated with the sequences disclosed herein.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a cell” or “a sample” includes a plurality of such cells or samples, respectively, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached exemplary claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The present invention relates to a biomarker for prostate cancer and method of using the same. More particularly, the invention relates to a method of using a membrane-associated C family G protein-coupled receptor GPRC6A as a biomarker for characterizing prostate cancer progression.

The present invention features a GPRC6A biomarker gene or gene products for diagnosing or prognosing prostate cancer in a sample. A biomarker gene or gene product is a gene of gene product that is objectively measured and evaluated as an indicator of a pathogenic process such as cancer to a pharmacologic response to a therapeutic intervention. A biomarker gene means nucleic acids such as DNA, CDNA, RNA and the related coding sequences and proteins such a gene products as described herein. The expression of a biomarker gene or gene product is modulated by a pathogenic process such as cancer. The gene or gene product of this invention includes those specifically disclosed herein and any coding sequences that are highly homologous to the coding sequences disclosed herein.

In some embodiments, the present invention discloses a method of characterizing a disease in a subject. The method includes determining the level of GPRC6A in a biological sample from the subject, and comparing level of GPRC6A biological sample to a reference, wherein the disease is characterized based on a measurable difference in the level of GPRC6A in the biological sample as compared to the reference. In some embodiments, the increased expression level of GPRC6A biomarker gene or gene products in said subject sample is indicative of the disease. Non-limiting examples of the disease include, but not limited to, primary prostate cancer, treatment-resistant prostate cancer, higher proliferation index CD133⁺ glioblastomas, and human myelod leukemia cell lines.

In some embodiments, the presently-disclosed invention further includes determining the amount of cyclic AMP in the biological sample from the subject and comparing the amount of cyclic AMP in the biological sample to a reference, wherein the disease is characterized based on a measurable difference in the amount of cyclic AMP in the biological sample as compared to the reference. In some embodiments, the disease is prostate cancer. Yet in some other embodiments, the prostate cancer includes treatment-resistant prostate cancer.

In some embodiments, the present invention determines and compares the expression levels of GPRC6A by isolating GPRC6A from the biological sample of the subject. The present invention further includes performing an in vitro assay on the GPRC6A. The levels of the GPRC6A marker in a sample of a subject can be determined by any method which is known in the art and not particularly limited. Examples of the in vitro assay include, but not limited to, immunoassay, histological or cytological assay, quantitative real-time PCR, and mRNA expression level assay.

As used herein, when a biomarker is identified by a “gene”, “gene symbol” or the like (such as GPRC6A), it should be recognized that the biomarker is a product of that gene. A gene product can include, for example, mRNA and protein. As such, biomarkers of the presently-disclosed subject matter include polynucleotides and polypeptides.

The term “gene” is used broadly to refer to any segment of DNA associated with a biological function. Thus, genes include, but are not limited to, coding sequences and/or the regulatory sequences required for their expression. Genes can also include non-expressed DNA segments that, for example, form recognition sequences for a polypeptide. Genes can be obtained from a variety of sources, including cloning from a source of interest or synthesizing from known or predicted sequence information, and can include sequences designed to have desired parameters.

In some embodiments the present invention relates to biomarker gene products such as proteins and fragments. The biomarker proteins of this invention include those specifically identified and allelic variants, substitutions and homologs.

The terms “gene product” are used interchangeably with “polypeptide”, “protein”, “peptide”, and “fragments” which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. Thus, exemplary polypeptides include gene products, naturally occurring proteins, homologs, orthologs, paralogs, fragments and other equivalents, variants, and analogs of the foregoing.

In addition, standard gene/protein nomenclature guidelines generally stipulate human gene name abbreviations are capitalized and italicized and protein name abbreviations are capitalized, but not italicized. Further, standard gene/protein nomenclature guidelines generally stipulate mouse, rat, and chicken gene name abbreviations italicized with the first letter only capitalized and protein name abbreviations capitalized, but not italicized. In contrast, the gene/protein nomenclature used herein when referencing specific biomarkers uses all capital letters for the biomarker abbreviation, but is intended to be inclusive of genes (including mRNAs and cDNAs) and proteins across animal species.

The “reference” can include, for example, a level of the biomarker in one or more samples from one or more individuals without the disease (e.g., negative control), or a level of the biomarkers in one or more samples from one or more individuals with the disease (positive control). In some embodiments, the reference includes a level of the one or more biomarkers in a sample from the subject taken over a time course. In some embodiments, the reference includes a sample from the subject collected prior to initiation of treatment for the disease and/or onset of the disease and the biological sample is collected after initiation of the treatment or onset of the disease.

In some embodiments, the reference can include a standard sample. Such a standard sample can be a reference that provides amounts of the biomarker at levels considered to be control levels. For example, a standard sample can be prepared with to mimic the amounts or levels of the biomarker in one or more samples (e.g., an average of amounts or levels from multiple samples) from one or more individuals without or with the disease of interest. In some embodiments the standard sample can be a reference that provides amounts of biomarker at levels considered to associated with a a responder or non-responder to treatment.

In some embodiments, the reference can include control data. Control data, when used as a reference, can comprise compilations of data, such as may be contained in a table, chart, graph, e.g., standard curve, or database, which provides amounts or levels of biomarker considered to be control levels. Such data can be compiled, for example, by obtaining amounts or levels of the biomarker in one or more samples (e.g., an average of amounts or levels from multiple samples) from one or more individuals without or without the disease.

The term “biological sample” as used herein refers to any body fluid or tissue associated with a prostate cancer. In some embodiments, for example, the biological sample can be a saliva sample, a blood sample, a serum sample, a plasma sample, a urine sample, or sub-fractions thereof.

The term “characterizing” comprises providing a diagnosis, prognosis and/or theranosis. The terms “diagnosing” and “diagnosis” as used herein refer to methods by which the skilled artisan can estimate and even determine whether or not a subject is suffering from a given disease or condition. As such, a diagnosis is inclusive of identifying a risk of a disease. The skilled artisan often makes a diagnosis on the basis of one or more diagnostic indicators, such as for example a biomarker (e.g., biomarker expression level, biomarker signature), the amount (including presence or absence) of which is indicative of the presence, severity, or absence of the condition.

The term “diagnosing” and “diagnosis” as used herein refer to methods by which the skilled artisan can estimate and even determine whether or not a subject is suffering from a given disease or condition. The skilled artisan often makes a diagnosis on the basis of one or more diagnostic indicators, such as for example a biomarker, the amount (including presence or absence) of which is indicative of the presence, severity, or absence of the condition.

Along with diagnosis, clinical disease “prognosis” is also an area of great concern and interest. It is important to know the stage and rapidity of advancement of the prostate cancer in order to plan the most effective therapy. If a more accurate prognosis can be made, appropriate therapy, and in some instances less severe therapy for the patient can be chosen. Measurement of biomarker levels disclosed herein can be useful in order to categorize subjects according to advancement of prostate cancer who will benefit from particular therapies and differentiate from other subjects where alternative or additional therapies can be more appropriate.

Making a prognosis or “prognosticating” can refer to predicting a clinical outcome (with or without medical treatment), selecting an appropriate treatment (or whether treatment would be effective), or monitoring a current treatment and potentially changing the treatment, based on the presence or level of one or more biomarkers in a sample. “Prognosticating” as used herein refers to methods by which the skilled artisan can predict the course or outcome of a condition in a subject. As such, “making a diagnosis” or “diagnosing”, as used herein, is further inclusive of determining a prognosis, which can provide for predicting a clinical outcome (with or without medical treatment), selecting an appropriate treatment (or whether treatment would be effective), or monitoring a current treatment and potentially changing the treatment, based on the measure of diagnostic biomarker levels disclosed herein.

The phrase “prognosis” or “prognosing” as used herein refers to methods by which the skilled artisan can predict the course or outcome of a condition in a subject. The terms do not refer to the ability to predict the course or outcome of a condition with 100% accuracy, or even that a given course or outcome is predictably more or less likely to occur based on the presence, absence or levels of test biomarkers. Instead, the skilled artisan will understand that the terms refer to an increased probability that a certain course or outcome will occur; that is, that a course or outcome is more likely to occur in a subject exhibiting a given condition, when compared to those individuals not exhibiting the condition. For example, in individuals not exhibiting the condition (e.g., not expressing the biomarker or expressing it at a reduced level), the chance of a given outcome may be about 3%. In certain embodiments, a prognosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, or about a 95% chance.

The skilled artisan will understand that associating a prognostic indicator with a predisposition to an adverse outcome is a statistical analysis. For example, a biomarker level (e.g., quantity of expression in a sample) of greater than a control level in some embodiments can signal that a subject is more likely to suffer from a prostate cancer than subjects with a level less than or equal to the control level, as determined by a level of statistical significance. Additionally, a change in marker concentration from baseline levels can be reflective of subject prognosis, and the degree of change in marker level can be related to the severity of adverse events. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Preferred confidence intervals of the present subject matter are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. When performing multiple statistical tests, e.g., determining differential expression of a panel of biomarker levels, p values can be corrected for multiple comparisons using techniques known in the art.

In other embodiments, a threshold degree of change in the level of a prognostic or diagnostic biomarker can be established, and the degree of change in the level of the indicator in a biological sample can simply be compared to the threshold degree of change in the level. A preferred threshold change in the level for markers of the presently disclosed subject matter is about 5%, about 10%, about 15%, about 20%, about 25%, about 30%, about 50%, about 75%, about 100%, and about 150%. In yet other embodiments, a “nomogram” can be established, by which a level of a prognostic or diagnostic indicator can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

G-Protein Receptor Coupled proteins (“GPRCs”) have been identified as a large family of G protein-coupled receptors in a number of species. These receptors share a seven transmembrane domain structure with many neurotransmitter and hormone receptors, and are likely to underlie the recognition and G protein mediated transduction of various signals.

GPRC6A represents a growing number of GPCRs that are upregulated in primary and metastatic cancers, where they promote tumor formation and cancer progression. Indeed, our data suggest that GPRC6A may provide a molecular mechanism to explain the associations between nutritional factors and prostate cancer risks. Activation of GPRC6A may also provide another mechanism to explain the effects of arginine deprivation therapy to affect cancer sensitivity. Another receptor closely related to GPRC6A, CASR, is also capable of sensing both cells was only partially inhibited by a dominant negative CASR construct, suggesting the possible presence of other mechanisms linking calcium and amino acids to prostate cancer growth. The effect of GPRC6A on prostate cell proliferation and migration may represent an accentuation in malignant cells of the physiological role of GPRC6A to integrate the response to nutrients and anabolic steroids with energy metabolism and responses of multiple tissues. GPRC6A potentially has both direct and indirect effects on prostate cancer. GPRC6A is a potent activator of ERK signaling and is a possible downstream signaling pathway whereby this receptor directly regulates prostate cancer growth. Activation of ERK has a central role in prostate cancer cell proliferation. Indeed, in vitro studies demonstrate that the growth factor induced proliferation of PC-3 cells requires ERK phosphorylation and treatment of PC-3 cells with PD98059, a chemical inhibitor of the ERK pathway, obliterates growth factor-mediated cell proliferation. GPCR-mediated proliferation may be particularly relevant in androgen-independent prostate cancer, since ERK phosphorylation is noted during carbachol treatment of androgen-independent PC-3 and DU145 cells but not in androgen-dependent LNCaP cells, GPRC6A might also have indirect effects to regulate prostate cancer through its effects on sex steroid metabolism. In this regard, it has been recently found that ablation of this orphan G-protein coupled receptor leads to undermasculinization associated with decreased muscle mass, increased adiposity, and low circulating testosterone and elevated estradiol levels in male mice, suggesting that GPRC6A may also modulate sex steroid end organ responses.

In some embodiments, the present invention provides a kit for detecting prostate cancer in a subject, including an agent that selectively binds to GPRC6A. The kit of the present invention comprises a substance for determining the level of GPRC6A gene or gene products as a prostate cancer marker (for example, RT-PCR primers). The kit includes an agent that selectively binds to a GPRC6A. In some embodiments, the agent is probe or primers. The kit of the present invention further comprises probes or primers to detect GPRC6A gene expression level. In some embodiments of the invention, the primers are selected from the group consisting of SEQ ID 1, 2, 12, 13, 14, 15, 16, 17, 18, 19.

Yet in some embodiments of the invention, the kit can include an antibody to detect GPRC6A biomarker gene products. Further, the kit can further contains reagents and/or other materials to measure the mRNA or protein level for the expression of GPRC6A. For example, the kit of the present invention can comprise a buffer (for dilution or washing), a standard antigen, a labeled antibody capable of immunologically reacting with an anti-GPRC6A antibody in a specific manner, a substrate reagent capable of causing color development, luminescence, or fluorescence, and an instruction describing procedures and an evaluation method. In some embodiments, the expression level of the GPRC6A biomarker gene or gene product is higher in prostate cancer tissue than in normal tissue.

As used herein, the term “selectively-bind” refers to an interaction between an agent and a binding site of a polypeptide or polynucleotide molecule. In some embodiments, the interaction between the agent and the binding site can be identified as “selective” if: the equilibrium dissociation constant (Kd) is about the same or less than the Kd of the agent and a reference polynucleotide or polypeptide binding site; the equilibrium inhibitor dissociation constant (Ki) is about the same or less than the Ki of an agent and a reference polynucleotide or polypeptide binding site; or the effective concentration at which binding of the agent is inhibited by 50% (EC50) is about the same or less than the EC50 of the agent and a reference polynucleotide or polypeptide binding site.

In some embodiments, the interaction between an agent and the binding site can be identified as “selective” when the equilibrium dissociation constant (Kd) is less than about 100 nM, 75 nM, 50 nM, 25 nM, 20 nM, 10 nM, 5 nM, or 2 nM. In some embodiments, the interaction between [substrate] and the binding site can be identified as “selective” when the equilibrium inhibitor dissociation constant (Ki) is less than about is less than about 100 μM, 75 μM, 50 μM, 25 μM, 20 μM, 10 μM, 5 μM, or 2 μM, when competing with glucose. In some embodiments, the interaction between [substrate] and the binding site can be identified as “selective” when the effective concentration at which [substrate] binding is inhibited by 50% (EC50) is less than about 500 μM, 400 μM, 300 μM, 100 μM, 50 μM, 25 μM, or 10 μM.

Some other embodiments of the present invention provide a method of treating treatment-resistant prostate cancer in a subject with said cancer, comprising administering to the subject a therapeutically effective amount of an androgenergic antagonist of GPRC6A. Examples of the treatment-resistant prostate cancer includes, but not limited to castration-resistant prostate cancer, and chemotherapy-resistant prostate cancer.

As used herein, the terms “treatment” or “treating” relate to curing or substantially curing a condition, as well as ameliorating at least one symptom of the condition, and are inclusive of prophylactic treatment and therapeutic treatment.

As would be recognized by one or ordinary skill in the art, treatment that is administered prior to clinical manifestation of a condition then the treatment is prophylactic (i.e., it protects the subject against developing the condition). If the treatment is administered after manifestation of the condition, the treatment is therapeutic (i.e., it is intended to diminish, ameliorate, control, or maintain the existing condition and/or side effects associated with the condition).

The terms relate to medical management of a subject with the intent to substantially cure, ameliorate, stabilize, or substantially prevent a condition of interest (e.g., disease, pathological condition, or disorder), including but not limited to prophylactic treatment to preclude, avert, obviate, forestall, stop, or hinder something from happening, or reduce the severity of something happening, especially by advance action.

As such, the terms treatment or treating include, but are not limited to: inhibiting the progression of a condition of interest; arresting or preventing the development of a condition of interest; reducing the severity of a condition of interest; ameliorating or relieving symptoms associated with a condition of interest; causing a regression of the condition of interest or one or more of the symptoms associated with the condition of interest; and preventing a condition of interest or the development of a condition of interest.

The terms includes active treatment, that is, treatment directed specifically toward the improvement of a condition of interest, and also includes causal treatment, that is, treatment directed toward removal of the cause of the condition of interest. In addition, the terms includes palliative treatment, that is, treatment designed for the relief of symptoms rather than the curing of the condition of interest; preventative treatment, that is, treatment directed to minimizing or partially or completely inhibiting the development of the associated condition of interest; and supportive treatment, that is, treatment employed to supplement another specific therapy directed toward the improvement of the associated condition of interest.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

The term “androgenergic antagonist” refers to agents that can prevent androgens from expressing their biological effects on responsive tissues. These agents alter the androgen pathway by blocking the appropriate receptors, competing for binding sites on the cell's surface, or affecting androgen production. Androgenergic antagonist can be prescribed to treat an array of diseases and disorders. In men, these agents are most frequently used to treat prostate cancer. In women, these agents are used to decrease levels of male hormones causing symptoms of hyperandrogenism. Androgenergic antagonist present in the environment have become a topic of concern. Many industrial chemicals, pesticides and insecticides exhibit antiandrogenic effects. Non-limiting examples of the androgenergic antagonist include, but not limited to, allylestrenol, oxendolone, osaterone acetate, bicalutamide, steroidal, anti-androgergic agents, medroxyprogesterone (MPA), cyproterone, cyproterone acetate (CPA), dienogest, flutamide, nilutamide, spironolactone, 5alpha-reductase inhibitors, dutasteride, finasteride, salts thereof, gold nanoparticles thereof, combinations thereof, and the like. In some embodiments of the present invention, examples of the androgenergic antagonist includes, but not limited to a gold nanoparticle of α-bicalutamide, or a gold nanoparticle of β-bicalutamide.

In some embodiments, the invention provides biomarker polynucleotides containing specific portions of the biomarker mRNA sequences, including those that are complementary to these sequences, such that they encode a biomarker protein and fragments thereof. Polynucleotides of this invention can be used to design molecules to inhibit the expression of a GPRC6A protein biomarker gene. For example, antisense molecules such as siRNAs can be developed to modulate the expression of the gene.

The terms “small interfering RNA”, “short interfering RNA”, “small hairpin RNA”, “siRNA”, and shRNA are used interchangeably and refer to any nucleic acid molecule capable of mediating RNA interference (RNAi) or gene silencing. See e.g., Bass, Nature 411:428-429, 2001; Elbashir et al., Nature 411:494-498, 2001a; and PCT International Publication Nos. WO 00/44895, WO 01/36646, WO 99/32619, WO 00/01846, WO 01/29058, WO 99/07409, and WO 00/44914. In one embodiment, the siRNA comprises a double stranded polynucleotide molecule comprising complementary sense and antisense regions, wherein the antisense region comprises a sequence complementary to a region of a target nucleic acid molecule (for example, a nucleic acid molecule encoding COMT, ADRB2, or ABRB3). In another embodiment, the siRNA comprises a single stranded polynucleotide having self-complementary sense and antisense regions, wherein the antisense region comprises a sequence complementary to a region of a target nucleic acid molecule. In another embodiment, the siRNA comprises a single stranded polynucleotide having one or more loop structures and a stem comprising self-complementary sense and antisense regions, wherein the antisense region comprises a sequence complementary to a region of a target nucleic acid molecule, and wherein the polynucleotide can be processed either in vivo or in vitro to generate an active siRNA capable of mediating RNAi. As used herein, siRNA molecules need not be limited to those molecules containing only RNA, but further encompass chemically modified nucleotides and non-nucleotides.

The presently disclosed subject matter takes advantage of the ability of short, double stranded RNA molecules to cause the down regulation of cellular genes, a process referred to as RNA interference. As used herein, “RNA interference” (RNAi) refers to a process of sequence-specific post-transcriptional gene silencing mediated by a small interfering RNA (siRNA). See Fire et al., Nature 391:806-811, 1998 and U.S. Pat. No. 6,506,559, each of which is incorporated by reference herein in its entirety. The process of post-transcriptional gene silencing is thought to be an evolutionarily conserved cellular defense mechanism that has evolved to prevent the expression of foreign genes (Fire, Trends Genet 15:358-363, 1999).

In some other embodiments of the present invention, the likelihood of a positive therapeutic effect of the androgenergic antagonist can be predicted by determining the amount of cyclic AMP in a biological sample from the subject before and after administration of the androgenergic antagonist. Non-limiting examples of the androgenergic antagonists includes, bicalutamide, gold nanoparticles of α-bicalutamide (α-Bic-AuNP), and gold nanoparticles of β-bicalutamide (β-Bic-AuNP).

In other embodiments of the present invention, gold nanoparticles of α-bicalutamide, and gold nanoparticle of β-bicalutamide can modulate GPRC6A and affect downstream cAMP production. In vitro studies demonstrated that cAMP second messenger accumulated in response to overnight stimulation of GPRC6A signal transduction by α-Bic-AuNP and β-Bic-AuNP in an androgen receptor(AR) null/GPRC6A and androgen receptor(AR) null/GPRC6A⁺ transfected cell line. Gold nanoparticles of α-bicalutamide and gold nanoparticles of β-bicalutamide significantly stimulated GPRC6A in an androgen competitive manner eliciting cAMP production at low micromolar ligand (sub-nanomolar gold nanoparticle) concentrations. (Dreaden, E. C., et al, Antiandrogen gold nanoparticles dual-target and overcome treatment resistance in hormone-insensitive prostate cancer cells, Bioconjugage Chem. 2012, 23, 1507-1512).

The following Examples have been included to illustrate modes of the presently disclosed subject matter. In light of the present disclosure and the general level of skill in the art, those of skill will appreciate that the following Example are intended to be exemplary only and that numerous changes, modifications, and alterations can be employed without departing from the scope of the presently disclosed subject matter.

### Example 1

The current investigations examined the expression and function of GPRC6A in prostate cancer progression both in vitro and in vivo using a combination of molecular and mouse genetic approaches. The current investigation found that GPRC6A is expressed in normal prostate and is increased in cancer-derived prostate cell lines. Bothe BSA-coupled testosterone and OC stimulates ERK phosphorylation in HEK-293 cells transfected with GPRC6A and in 22RV1 and PC-3 human prostate cancer cell expressing endogenous GPRC6A. Moreover, RNAi-mediated knockdown of GPRC6A in prostate cancer cells inhibits ligand-stimulated proliferation and chemotaxis in vitro. Finally, deletion of Gprc6a in the TRAMP mouse model of prostate cancer significantly retarded prostate cancer progression and improved survival. Based on these findings it is proposed that GPRC6A may mediate the prostate response to nutrients, OC and non-genomic effects of androgens and that targeting this receptor with an antagonist may provide a complementary strategy to treat androgen resistant prostate cancer.

Materials and Methods

Reagents and Cell Culture

Testosterone 3-(O-carboxymethyl) oxime-BSA, L-Arginine, calcium chloride, and zinc chloride were obtained from Sigma-Aldrich. In the case of testosterone-BSA, the freshly purified testosterone was used in each experiment. Before each experiment, stock solutions of BSA conjugates were mixed with dextran (0.05 mg/ml) and charcoal (50 mg/ml) for 30 min, centrifuged at 3000×g for 10 min, and passed through a 0.22-mm pore size filter to remove any potential contamination with free testosterone. Methytrienolone (R1881) was purchased from Perkin-Elmer. Osteocalcin (purified from bovine bone) was purchased from Biodesign International. Total human RNAs were obtained from Clontech.

The prostate cancer cell lines 22Rv1, PC-3 and LNcaP and the prostate cell lined RWPE-1 derived from normal prostate were obtained from the American Type Culture Collection (Manassas, Va.). The prostate cells were maintained in RPMI 1640 supplemented with 10% fetal bovine serum (Gibco Life Technologies, Inc.). Cells (10³ cells/well) were cultured in triplicate in a 96-well flat-bottomed microculture dish using RPMI 1640 containing 10% CFBS in the presence and absence of various concentrations of GPRC6A ligands, including: calcium, amino acids, calcimimetic and OC for 72 h. Cell proliferation was determined by counting cells with a hemocytometer (21).

RT-PCR and Real-Time RT-PCR

Human tissue cDNAs were obtained from Clontech Laboratories, Inc. Total RNA from human prostate cancer cells was isolated with RNeasy Mini Kit (Qiagen Inc., Valencia, Calif.). RT-PCR was done using two-step RNA PCR (Perkin-Elmer). In separate reactions, 2.0 μg of DNase-treated total RNA was reverse-transcribed into cDNA with the respective reverse primers specified below and Moloney murine leukemia virus reverse transcriptase (Gibco Life Technologies, Inc.). Reactions were carried out at 42° C. for 60 min followed by 94° C. for 5 min and 5° C. for 5 min. The products of first-strand cDNA synthesis were directly amplified by PCR using AmpliTaq DNA polymerase (Perkin-Elmer). The primers for human GPRC6A are as follows: hGPRC6A.F203: caggagtgtgttggctttga (SEQ ID NO: 1) and hGPRC6A.R630: atcaggtgagccattgcttt (SEQ ID NO: 2). For housekeeping gene control G3PDH gene, G3PDH.F143: gaccccttcattgacctcaactaca (SEQ ID NO: 3) and G3PDH.R1050: ggtcttactccttggaggccatgt (SEQ ID NO: 4). For quantitative real time RT-PCR assessment of PSA and Runx 2II genes expression, total RNA was isolated and reverse transcribed from 22Rv1 cells that stimulated with or without OC (80 ng/ml), arginine (30 mM) and R1881 (100 nM) as previously described using specific primer sets. The primers sequences were PSA. For: ttggaaatgaccaggccaag (SEQ ID NO: 5) and PSA.Rev: agcaaccctggacctcacac (SEQ ID NO: 6); Runx 2. For: attcctgtagatccgagcacc (SEQ ID NO: 7) and Runx 2. Rev: gctcacgtcgctcattttgc (SEQ ID NO: 8), and the threshold cycle (Ct) of tested-gene product from the indicated genotype was normalized to the Ct for cyclophilin A as previously described (18).

Measurement of Total and Phospho-ERK by Western-Blot Analysis

Briefly, human prostate cancer cells and the cells transfected with GPRC6A will be made quiescent by overnight incubation in serum-free DMEM/F12 containing 0.1% BSA and stimulated with various ligands at different doses. ERK activation will be assessed 5 to 30 minutes after treatment by immunoblotting using anti-phospho-ERK1/2 MAP kinase antibody (Cell Signaling Technology) corrected for the amount of ERK using an anti-ERK1/2 MAP Kinase antibody (Cell Signaling Technology) to measure ERK levels.

siRNA Suppression of GPRC6A Gene Expression.

For GPRC6A knockdown experiments, two short interfering RNAs (siRNAs) (19 nucleotides each) have been designed from the hGPRC6A sequence (NM_{—}148963) (SEQ ID NO: 9). These are GPRC6A siRNA-202: CCAGGAGTGTGTTGGCTTT (SEQ ID NO: 10) and siRNA-514: GCCACAGGTGGGTTATGAA (SEQ ID NO: 11). Two siRNA hairpins were synthesized and cloned into a pSilencer™ 4.1-CMV neo vector (Ambion). A circular pSilencer™ 4.1-CMV neo vector that expresses a hairpin siRNA with limited homology to any known sequence was used as a negative control. The constructs of siRNA duplexes have been stably transfected into human prostate cancer cells using Lipofectamine™ (Invitrogen) and were selected by G418 (Invitrogen). Successful knock down of GPRC6A will be confirmed by assessing RT-PCR analysis of GPRC6A expression.

Chemotaxis Assay.

The migration of 22Rv1 cells stably expressing negative control siRNA and human GPRC6A-specific siRNA using a previously described chemotaxis assay (22). The migration index was calculated, and was defined as number of cells crossing the filter toward calcium or OC (various concentrations)/number of cells migrating toward medium alone (control). Each experiment was performed at least three times, in duplicate.

Mouse Models

Mice were maintained and used in accordance with recommendations as described (National Research Council. 1985; Guide for the Care and Use of Laboratory Animals DHHS Publication NIH 86-23, Institute on Laboratory Animal Resources, Rockville, Md.) and following guidelines established by the University of Tennessee Health Science Center Institutional Animal Care and Use Committee. The Gprc6a-deficient mouse model was created by replacing exon 2 of the Gprc6a gene with the hygromycin resistance gene (18). TRAMP transgenic mouse was purchased from The Jackson Laboratory (Stock #: 003135). Transfer of Gprc6a deficiency onto the TRAMP background, male Gprc6a^{-/-} mice was first crossed with female TRAMP to generate Gprc6a^{+/-}/TRAMP mice. Then, male Gprc6a^{+/-} mice was crossed with female Gprc6a^{+/-}/TRAMP mice. For genotyping Gprc6a deficiency mice, the PCR primers are Athx-1: gaataactagcaggaggggcgctggaaggag (SEQ ID NO: 12) and Athx-2: cagagtggcagccattgctgctgtgacttcg (wild type pair) (SEQ ID NO: 13); Athx-F: cacgagagatcgtggggtatcgacagag (SEQ ID NO: 14) and Athx-R: ctacatggcgtgatttcatatgcgcgattgctg (knockout pair) (SEQ ID NO: 15). For genotyping TRAMP transgenic mice, the PCR primers are oIMR0015: caaatgttgcttgtctggtg (SEQ ID NO: 16) and oIMR0016: gtcagtcgagtgcacagttt (wild type pair) (SEQ ID NO: 17); oIMR0068: cagagcagaattgtggagtgg (SEQ ID NO: 18) and oIMR0069: ggacaaaccacaactagaatgcagtg (Transgene pair) (SEQ ID NO: 19).

Statistics

Differences between groups are evaluated by one-way analysis of variance. All values are expressed as means±SEM. All computations were performed using the Statgraphic statistical graphics system (STSC Inc.).

Results:

Detection of GPRC6A mRNA in Human Prostate Cancer Cell Line and Prostate Cancer Tissues.

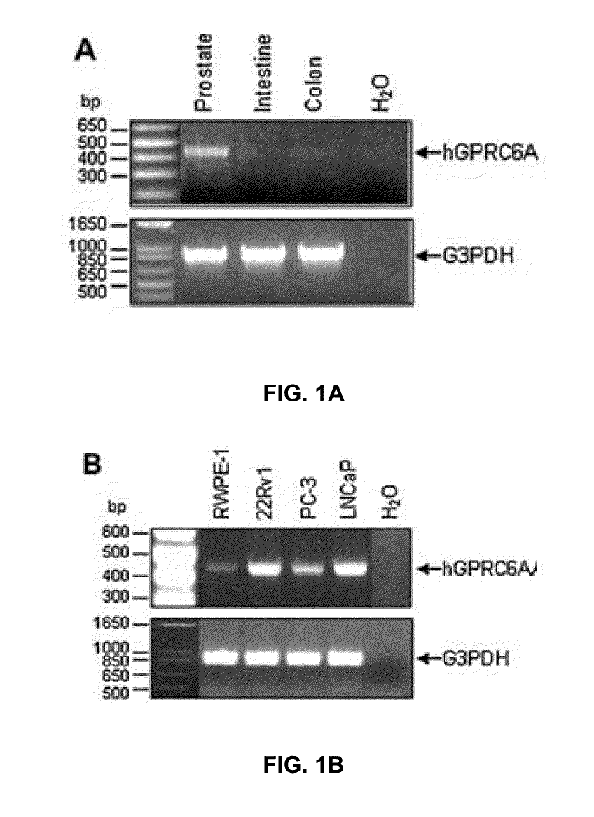

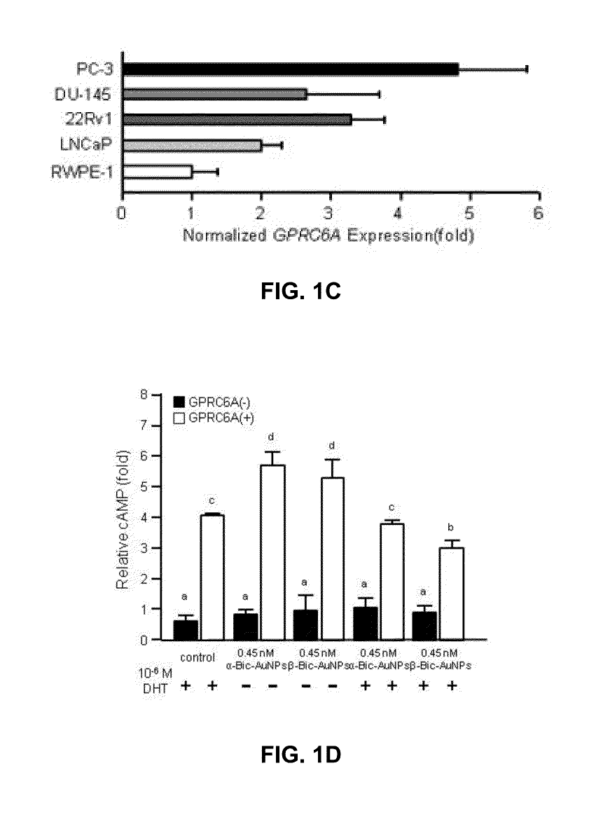

Now referring to FIG. 1 which shows that GPRC6A is over-expressed in human prostate cancer cell lines and human prostate cancer tissue. RT-PCR was performed with intron-spanning primers specific for human GPRC6A in human prostate tissue (FIG. 1A) and human prostate cell lines RWPE-1, 22Rv1, PC-3 and LNCaP (FIG. 1B). FIG. 1A shows GPRC6A expression in normal human prostate gland. PCR products were amplified from normal human multiple tissue cDNAs: prostate, intestine and colon using human specific intron-spanning primers. FIG. 1B shows GPRC6A over-expression of human prostate cancer cell lines by RT-PCR. RWPE-1 is human prostate epithelial cell. 22Rv1, PC-3 and LNCaP are human prostate cancer cell lines. The primers for GPRC6A application are described in Methods. House-keeping control gene glyceraldehyde-3-phosphate dehydrogenase (G3PDH) was used for a positive control of RNA integrity. A product of the predicted size, 428 bp in all prostate cells and prostate tissue was amplified. The gene products from 22Rv1, PC-3 and LNCaP were identified as human GPRC6A by DNA sequence analysis. The level of GPRC6A expression was higher in prostate cancer cell lines, 22Rv1, PC-3 and LNCaP than in normal human prostate cell line RWPE-1 (FIG. 1B). To explore GPRC6A expression in prostate cancer tissues, the Gene Expression Omnibus (GEO) database was queried using the search terms GPRC6A and prostate (http://www.ncbi.nlm.nih.gov/sites/entrez?db=geo). GPRC6A was found to be up-regulated in primary prostate cancer (GEO accession GDS1439) (23). GPRC6A is also highly expressed in other cancers, including higher proliferation index CD133⁺ glioblastomas (GEO accession GDS2728) (24), and human myeloid leukemia cell lines (GEO accession GDS2251) (25). Together these data suggest a potential role of GPRC6A in malignant transformation of the prostate cancer and other cancers. FIG. 1C shows Upregulated GPRC6A mRNA expression levels measured from various prostate cancer cell lines relative to non-malignant RWPE-1 prostate cells (See Supporting Information). Downstream production of cyclic adenosine monophosphate (cAMP) in response to GPRC6A stimulation was assessed using an established AR⁻/GPRC6A⁻ and AR⁻/GPRC6A⁺ transfected cell line (See Supplementary Information).³³ α-Bic- and β-Bic-AuNPs significantly stimulated GPRC6A in an androgen-competitive manner (FIG. 1D), eliciting cAMP production at sub-nM concentrations. FIG. 1D is Androgen-competitive downstream production of cyclic adenosine monophosphate (cAMP) accumulated in response to overnight GPRC6A stimulation by α-Bic- and β-Bic-AuNPs in an AR⁻/GPRC6A⁻ and AR⁻/GPRC6A⁺ transfected cell line. DHT, dihydrotestosterone. Error bars represent SEM. P for individual values relative to untreated controls or as indicated; *P<0.05, **P<0.01.

Effects of Calcium, OC, and Arginine on ERK Activity, Cell Number and Gene Expression in Human Prostate Cancer Cell Lines.

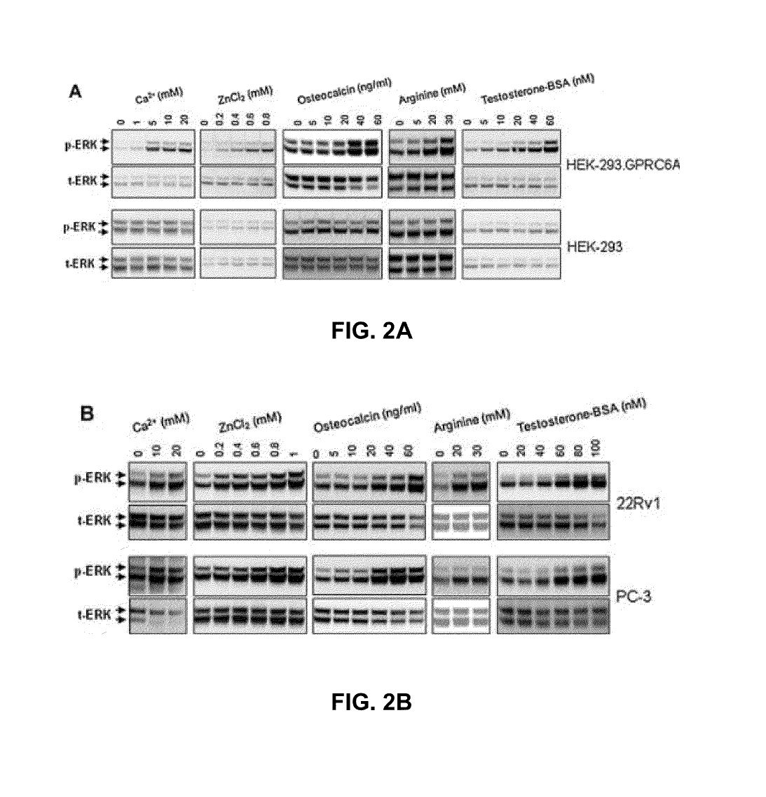

Referring to FIG. 2. FIG. 2A shows Dose-dependent effects of extracellular calcium, zinc, OC, arginine, and testosterone-BSA on GPRC6A-mediated EKR activation in HEK cells transfected with GPRC6A. Here, calcium (1-20 mM), zinc (0.2-0.8 mM), OC (5-60 ng/ml), arginine (5-30 mM), and testosterone-BSA (5-100 nM) are confirmed to be functional ligands for GPRC6A (16-17, 19). Calcium, zinc, OC, arginine, and testosterone resulted in a dose-dependent stimulation of ERK activity in HEK-293 cells overexpressing GPRC6A, whereas non-transfected HEK-293 cells failed to respond to any of these ligands. The effects of these GPRC6A ligands on ERK activation in androgen receptor positive 22Rv1 and androgen receptor negative PC-3 cells were examined in FIG. 2B which shows dose-dependent effects of extracellular calcium, zinc, OC, arginine and testosterone on ERK activation in human prostate cancer cell lines 22Rv1 and PC-3. Calcium (10 mM), zinc (0.2 mM), OC (20 ng/ml), arginine (20 mM), and testosterone-BSA (60 nM) increased phospho-ERK in both of these cells. The HEK cells transfected with GPRC6A or without the plasmid cDNA of GPRC6A or 22Rv1 or PC-3 were incubated in Dulbecco's modified Eagle's medium/F-12 containing 0.1% bovine serum albumin quiescence media and exposed to the extracellular calcium, OC, arginine, or testosterone-BSA at indicated concentrations for 5 min, and ERK activation was determined as described under Materials and Methods. Representative blots are shown, and the results were verified in at least three independent experiments.

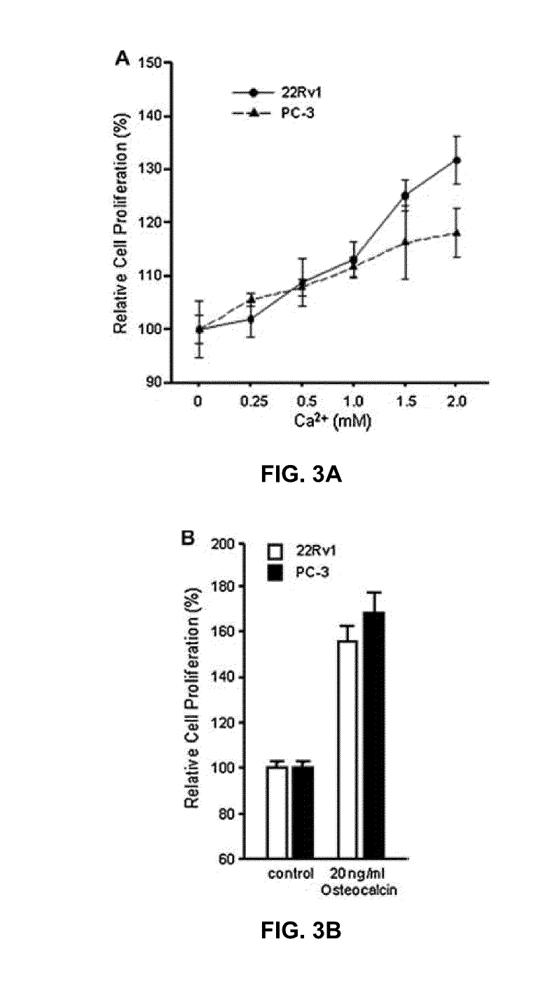

Now referring to FIG. 3. To determine if GPRC6A ligands enhance prostate cell growth, changes were accessed in cell numbers in 22Rv1 and PC-3 cells exposed to different concentrations of extracellular calcium or OC over a three-day culture period. Human prostate cancer cells, 22Rv1 or PC-3 (10³ cells/well) grown under subconfluent conditions were cultured in triplicate in a 96-well flat-bottomed microculture dish using RPMI 1640 containing 10% CFBS with various concentrations of GPRC6A ligands: calcium FIG. 3A and OC FIG. 3B for 72 h. Cell proliferation was determined by counting cells in a hemocytometer method as described in Materials and methods. In all of the above studies, values for relative cell proliferation (expressed as percent of control) represent the mean±SEM of a minimum of three separate experiments. * indicates a significant difference from control and stimulation at p<0.05, respectively. Control 22Rv1 and PC-3 cells typically increased their cell number ˜5-fold, respectively, in the absence of added ligands over this time period. The addition of calcium to the medium modestly increased the number of cells by ˜30% (FIG. 3A), whereas OC resulted in a ˜70% increase in cell number (FIG. 3B).

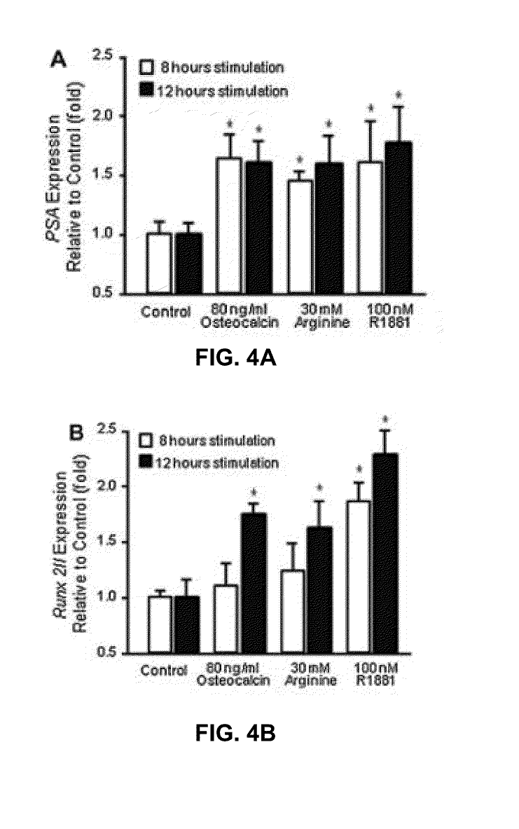

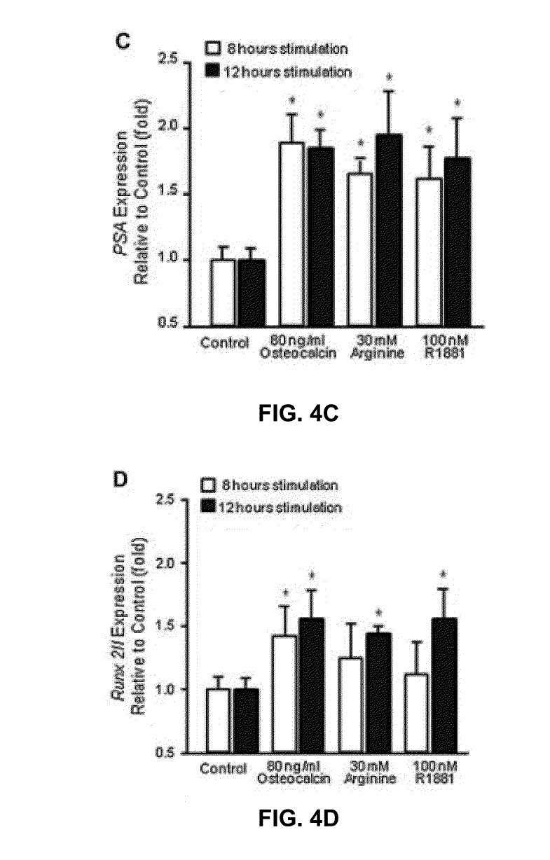

Now referring to FIG. 4, FIG. 4 shows the ligands of GPRC6A stimulated human prostate cancer cells expression of PSA and Runx2 in 22Rv1 and PC-3 cells. After 8 hours stimulation, OC, arginine and R1881 stimulated PSA and Runx 2II gene expression in human prostate cancer 22Rv1 (FIG. 4A and FIG. 4B) and PC-3 cells (FIG. 4C and FIG. 4D). * indicates a significant difference from control and stimulation at p<0.05 respectively.

Human Prostate Cancer Cell Lines 22Rv1 and PC-3 Respond to Extracellular Calcium, OC and Arginine Through GPRC6A.

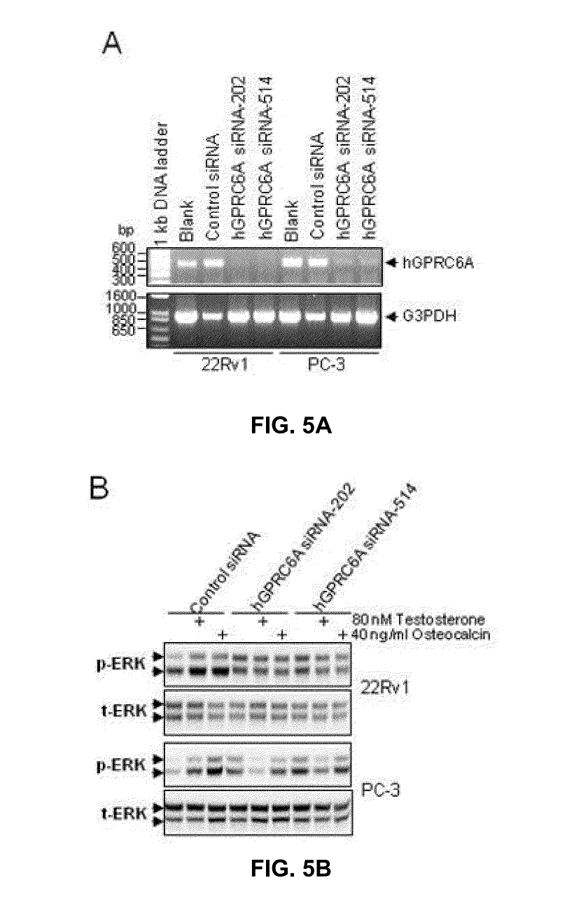

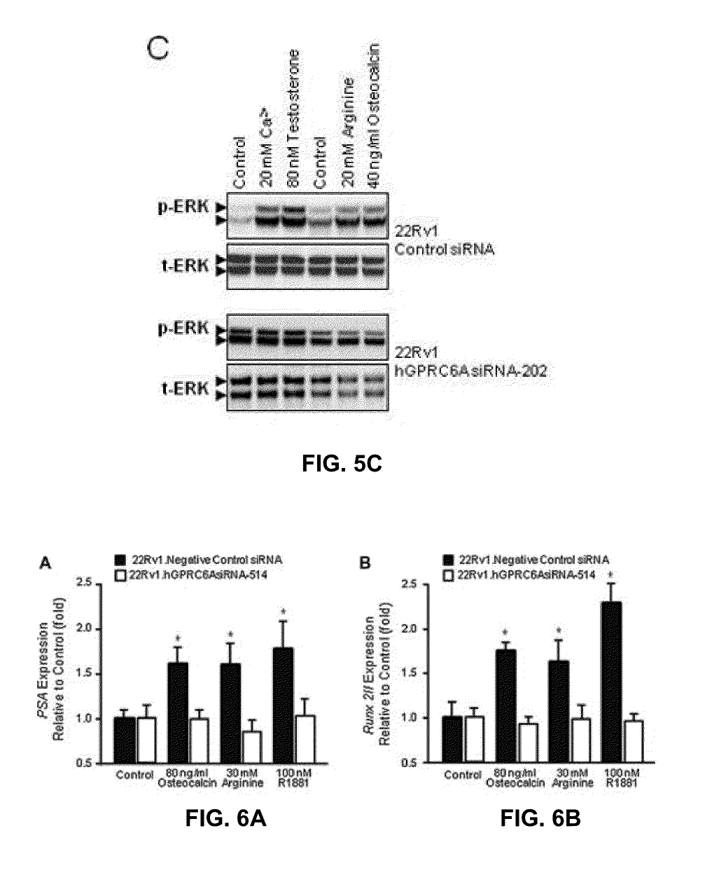

Now referring to FIG. 5, FIG. 5 shows GPRC6A siRNAs inhibited GPRC6A-mediated activation of phosph-ERK in human prostate cancer cell lines. To confirm the importance of GPRC6A signaling in prostate cancer cell line, the response to GPRC6A ligands was accessed after knock-down of GPRC6A using siRNA. For these studies, ERK activity was assessed in 22Rv1 and PC-3 cells stably transfected with GPRC6A siRNA-202 and siRNA-514. These responses were compared control groups consisting of mock-transfected cells and transfected cells with a random negative control siRNA plasmid. Decreased levels of mRNA expression of GPRC6A was observed in 22Rv1 or PC-3 cells transfected with interfering RNAs, GPRC6A siRNA-202 and siRNA-514, compared to controls (FIG. 5A). OC and testosterone stimulated phospho-ERK activity was significantly decreased in both 22Rv1 and PC-3 human prostate cancer cell transfected with GPRC6A siRNA-202 and siRNA-514 (FIG. 5B). In addition, the current studies found that extracellular calcium, testosterone, arginine and OC failed to stimulate ERK phosphorylation in 22Rv1 cells transfected with GPRC6A siRNA-202 (FIG. 5C).

GPRC6A Regulation of PSA and RUNX2.

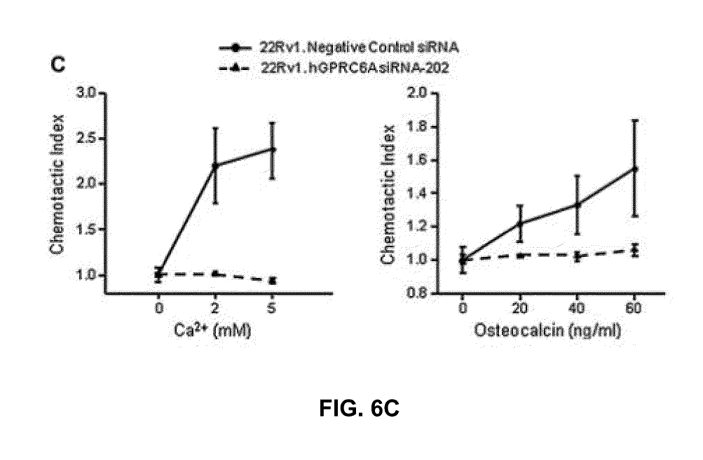

To investigate whether the observed ligand-induced PSA and Runx2 gene expression in the prostate cancer cells is due to simulation of GPRC6A, the response to GPRC6A ligands was examined after knock-down of GPRC6A. ERK activity was assessed in 22Rv1 stably transfected with GPRC6A siRNA-514 and compared this response to transfected cells with a random negative control siRNA plasmid, which were used as controls. Significantly decreased levels of mRNA expression of PSA and Runx2 was observed (FIGS. 6A and B). These results suggest that endogenous GPRC6A accounts for the effects of calcium, OC and arginine to stimulate gene expression of PSA and Runx2 in human prostate cancer cell lines.

GPRC6A-Mediated Human Prostate Cancer 22Rv1 Calcium- and OC-Induced Cell Chemotaxis.

Now referring to FIG. 6. To assess whether GPRC6A mediates prostate cancer cells chemotaxis and metastases, the ability of calcium and OC to evoke chemotaxis of human prostate cancer cell 22Rv1 expressing GPRC6A was examined. The ligands of GPRC6A, OC, arginine and R1881 stimulated gene expression of PSA (FIG. A) and Runx2 (FIG. B) were attenuated by GPRC6A siRNA in 22Rv1, human prostate cancer cells. As shown in FIG. 6C, calcium and OC induced the chemotaxis of 22Rv1 prostate cancer cells (FIG. 6C). siRNA mediated inhibition of GPRC6A expression eliminated the ability of calcium and OC to evoke chemotaxis of the prostate cancer cells (FIG. 6C). The experiments were described in Materials and methods. In all of the above studies, values for relative cell proliferation (expressed as percent of control) represent the mean±SEM of a minimum of three separate experiments.

Effects of Superimposed Gprc6a Deficiency in the TRAMP Mouse Prostate Cancer Model.

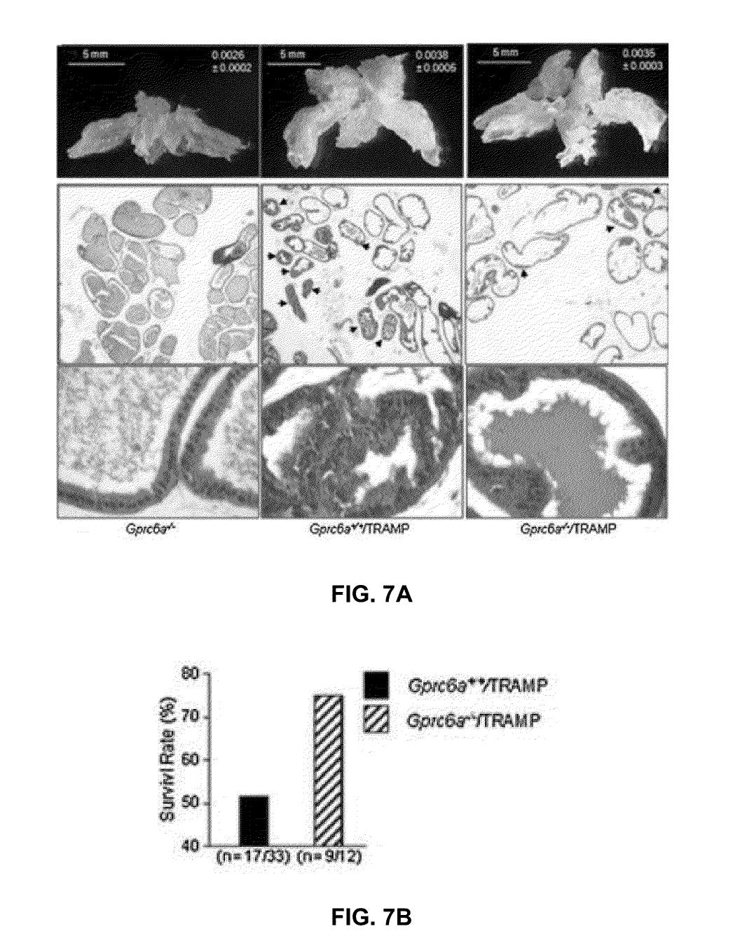

Now referring to FIG. 7, which shows the effects of superimposed Gprc6a deficiency in the TRAMP mouse. Finally, to examine the effects of GPRC6A on regulation of prostate cancer cell function in vivo, Gprc6a^{-/-} mice were intercrossed onto the TRAMP mouse model of prostate cancer. FIG. 7A shows the gross appearance of whole prostatic glands (Upper panel) and hematoxylin/eosin stained histological sections of ventral prostate (Middle panel, X5 magnification; Lower panel, X20 magnification) from Gprc6a^{-/-}, TRAMP and Gprc6a^{-/-}/TRAMP mice at 30 weeks-of-age. Consistent with prior reports (26), 30 week-old TRAMP mice had evidence of intraepithelial hyperplasia in the ventral prostate (FIG. 7A, middle panel)), whereas, Gprc6a^{-/-} mice had smaller but normal appearing prostate histology (FIG. 7A, left panel). Loss of Gprc6a in TRAMP mice resulted in markedly reduced intraepithelial hyperplasia (FIG. 7A, right middle and lower panels). In addition, a greater percentage of combined Gprc6a^{-/-}/TRAMP mice survived for 40 weeks compared to TRAMP mice with intact Gprc6a (75 vs 52%, respectively) (FIG. 7B). Values (inset, upper panel) represent Mean±SEM of prostate gland weights/body weights. Arrow (Lower panel) shows intraepithelial hyperplasia. (B) Comparison of the survival rates in TRAMP and compound Gprc6a^{-/-}/TRAMP mice.

Links between environmental factors and prostate cancer risk and progress have been described, but the molecular targets mediating these effects are not known. The main objective of our study was to determine if GPRC6A, a recently characterized nutrient, OC and androgen-sensing GPCR, plays a role in the pathogenesis of prostate cancer. Consistent with an important role of GPRC6A in prostate cancer growth and progression, the present invention illustrated that GPRC6A, which is expressed in normal prostate tissue and cells at low levels, is markedly elevated in prostate cancer tissue and cells. GPRC6A was over-expressed in human prostate cancer cell lines, 22Rv1, LNCap and PC-3 cells, as well as in human prostate cancer tissues. The present invention also demonstrated that the functional responses of prostate cancer cells to extracellular cations, OC and amino acids are mediated by GPRC6A, as evidenced by the ability of a wide range of agonists, including extracellular calcium, zinc, OC, testosterone and arginine, to stimulate GPRC6A-mediated ERK activation, cell proliferation, chemotaxis and gene expression in prostate cancer cells 22Rv1 and PC-3. In addition, the present invention established that the responses to GPRC6A agonists in prostate cancer cells in vitro were blocked by transfection with siRNAs against GPRC6A. Finally, the in vivo relevance of GPRC6A-signaling in prostate cancer was demonstrated by the finding that ablation of Gprc6a in TRAMP mice improved survival and decreased prostate cell hyperplasia. Together these data suggest a potential role of GPRC6A in malignant transformation of the prostate.

GPRC6A represents a growing number of GPCRs that are upregulated in primary and metastatic cancers, where they promote tumor formation and cancer progression. Indeed, our data suggests that GPRC6A may provide a molecular mechanism to explain the associations between nutritional factors and prostate cancer risks. Activation of GPRC6A may also provide another mechanism to explain the effects of arginine deprivation therapy to affect cancer sensitivity. Another receptor closely related to GPRC6A, CASR, is also capable of sensing both amino acids and calcium, but not osteoclacin, and is associated with prostate cancer progression. In this regard, CASR is expressed in human-derived prostate cancer cell lines, its expression is associated with metastatic prostate cancer, and extracellular calcium stimulates proliferation and PTHrp secretion in prostate cancer cell lines. However, calcium-mediated stimulation of PTHrp release from prostate cancer cells was only partially inhibited by a dominant negative CASR construct, suggesting the possible presence of other mechanisms linking calcium and amino acids to prostate cancer growth.

GPRC6A's effect on prostate cell proliferation and migration may represent an accentuation in malignant cells of the physiological role of GPRC6A to integrate the response to nutrients and anabolic steroids with energy metabolism and responses of multiple tissues (18-19). GPRC6A potentially has both direct and indirect effects on prostate cancer. GPRC6A is a potent activator of ERK signaling and is a possible downstream signaling pathway whereby this receptor directly regulates prostate cancer growth. Activation of ERK has a central role in prostate cancer cell proliferation. Indeed, in vitro studies demonstrate that the growth-factor-induced proliferation of PC-3 cells requires ERK phosphorylation and treatment of PC-3 cells with PD98059, a chemical inhibitor of the ERK pathway, obliterates growth-factor-mediated cell proliferation. GPCR-mediated proliferation may be particularly relevant in androgen-independent prostate cancer, since ERK phosphorylation is noted during carbachol treatment of androgen-independent PC-3 and DU145 cells but not in androgen-dependent LNCaP cells, GPRC6A might also have indirect effects to regulate prostate cancer through its effects on sex steroid metabolism. In this regard, it was recently found that ablation of this orphan G-protein coupled receptor leads to undermasculinization associated with decreased muscle mass, increased adiposity, and low circulating testosterone and elevated estradiol levels in male mice, suggesting that GPRC6A may also modulate sex steroid end organ responses.

There are several implications of our findings. First, the increased expression of GPRC6A in prostate cancer supports the genome wide associative studies linking the GPRC6A locus to prostate cancer in Japanese males and may identify a new biological marker associated with worse outcomes. Further studies are needed to define how usefully GPRC6A might be as a marker for prostate cancer by establishing the relationship between GPRC6A expression, tumor grade and outcomes. Second, our finding that GPRC6A modulates prostate cancer progression raises the possibility that that disruption of GPRC6A will have a positive benefit to halt prostate cancer progression. If so, development of antagonists to GPRC6A could potentially lead to alternative strategies to treat prostate cancer. Moreover, GPRC6A may be a novel target that can be used for the selective elimination of possibly more aggressive prostate cancer cells independently of the functional status of the intracellular androgen receptor. Third, the ability of GPRC6A to mediate the non-genomic effects of testosterone raises interesting questions about the inter-relationship between GPRC6A and nuclear androgen receptors in prostate cancers resistant to inhibition of AR. While current data support continued AR expression and function in castrate-resistant prostate cancer tumors, our data raise the possibility that GPRC6A might mediate some of the effects of androgens on prostate progression. Activation of GPRC6A stimulates PSA, which is also an AR target, and increases Rimx2, which is known to participate in epithelial-mesenchymal transition and prostate cancer metastasis. Additional studies will be needed to determine if GPRC6A represents the putative GPRC mediating the rapid response to androgens in prostate cancer cells and its relationship to the potential targeting of the nuclear AR to the plasma membrane.

The present invention also provide a potential explanation for the propensity of prostate cancer to metastasize to bone via the effects of GPRC6A to sense OC, which in turn promotes the ability of prostate cancer cells to colonize, grow, and survive in the bone microenvironment. Indeed, calcium and OC stimulation of chemotaxis in 22Rv1 human prostate cells in vitro and the fact that knockdown of the GPRC6A by siRNA inhibited extracellular calcium and OC-induced migration suggests that GPRC6A may function as a calcium and/or OC-sensor that mediates prostate cancer cell migration toward the calcium and OC-rich bone microenvironment.

In conclusion, the present invention suggests that the integrative physiological function of GPRC6A to coordinate nutrient, OC and anabolic steroids actions on a variety of tissues is exploited in prostate cancer cells to regulate prostate cancer progression in vitro and in vivo. Increased expression of GPRC6A in prostate cancer cells may increase the susceptibility to develop prostate cancer and stimulate its progression by mediating the cell proliferation and migration to bone in response responses to a wide variety of ligands, including calcium, OC, and sex steroids. In addition, developing drugs to antagonize GPRC6A may provide novel strategies to prevent diagnose and treat prostate cancer. Regardless, the increased expression of GPRC6A in prostate cancer could potential be a diagnostic marker, a prognostics indicator and a potential therapeutic target. Thus, GPRC6A represents a new target in prostate cancer research. Further studies are needed to establish the role of GPRC6A in pathogenesis and treatment of human prostate cancer.

### REFERENCE

Throughout this document, various references are mentioned. All such references are incorporated herein by reference, including the references set forth in the following list:
1. Sadar M D, Hussain M, Bruchovsky N. Prostate cancer: molecular biology of early progression to androgen independence. Endocr Relat Cancer. 1999; 6:487-502.
2. Ornish D, Magbanua M J, Weidner G, Weinberg V, Kemp C, Green C, et al. Changes in prostate gene expression in men undergoing an intensive nutrition and lifestyle intervention. Proc Natl Acad Sci USA. 2008; 105:8369-74.
3. Allen N E, Key T J, Appleby P N, Travis R C, Roddam A W, Tjonneland A, et al. Animal foods, protein, calcium and prostate cancer risk: the European Prospective Investigation into Cancer and Nutrition. Br J. Cancer. 2008; 98:1574-81.
4. Giovannucci E, Liu Y, Willett W C. Cancer incidence and mortality and vitamin D in black and white male health professionals. Cancer Epidemiol Biomarkers Prey. 2006; 15:2467-72.
5. Butler L M, Wong A S, Koh W P, Wang R, Yuan J M, Yu M C. Calcium intake increases risk of prostate cancer among Singapore Chinese. Cancer Res. 2010; 70:4941-8.
6. Ahn J, Albanes D, Peters U, Schatzkin A, Lim U, Freedman M, et al. Dairy products, calcium intake, and risk of prostate cancer in the prostate, lung, colorectal, and ovarian cancer screening trial. Cancer Epidemiol Biomarkers Prey. 2007; 16:2623-30.
7. Wu H C, Lin C C, Chen W C, Chen H Y, Tsai F J. Osteocalcin gene HindIll C/T polymorphism is a biomarker for prostate cancer and responsiveness to hormone therapy. Eur Urol. 2003; 43:197-200.
8. Huang W C, Xie Z, Konaka H, Sodek J, Zhau H E, Chung L W. Human osteocalcin and bone sialoprotein mediating osteomimicry of prostate cancer cells: role of cAMP-dependent protein kinase A signaling pathway. Cancer Res. 2005; 65:2303-13.
9. Pratap J, Lian J B, Stein G S. Metastatic bone disease: Role of transcription factors and future targets. Bone. 2010.
10. Goldenberg S L, Bruchovsky N, Rennie P S, Coppin C M. The combination of cyproterone acetate and low dose diethylstilbestrol in the treatment of advanced prostatic carcinoma. J. Urol. 1988; 140:1460-5.
11. Kampa M, Kogia C, Theodoropoulos P A, Anezinis P, Charalampopoulos I, Papakonstanti E A, et al. Activation of membrane androgen receptors potentiates the antiproliferative effects of paclitaxel on human prostate cancer cells. Mol Cancer Ther. 2006; 5:1342-51.
12. Scher H I, Sawyers C L. Biology of progressive, castration-resistant prostate cancer: directed therapies targeting the androgen-receptor signaling axis. J Clin Oncol. 2005; 23:8253-61.
13. Wellendorph P, Brauner-Osborne H. Molecular cloning, expression, and sequence analysis of GPRC6A, a novel family C G-protein-coupled receptor. Gene. 2004; 335:37-46.
14. Kuang D, Yao Y, Lam J, Tsushima R G, Hampson D R. Cloning and characterization of a family C orphan G-protein coupled receptor. J. Neurochem. 2005; 93:383-91.
15. Sirotnak F M, She Y, Khokhar N Z, Hayes P, Gerald W, Scher H I. Microarray analysis of prostate cancer progression to reduced androgen dependence: studies in unique models contrasts early and late molecular events. Mol Carcinog. 2004; 41:150-63.
16. Wellendorph P, Hansen K B, Balsgaard A, Greenwood J R, Egebjerg J, Brauner-Osborne H. Deorphanization of GPRC6A: a promiscuous L-alpha-amino acid receptor with preference for basic amino acids. Mol Pharmacol. 2005; 67:589-97.
17. Pi M, Faber P, Ekema G, Jackson P D, Ting A, Wang N, et al. Identification of a novel extracellular cation-sensing G-protein-coupled receptor. J Biol Chem. 2005; 280:40201-9.
18. Pi M, Chen L, Huang M Z, Zhu W, Ringhofer B, Luo J, et al. GPRC6A null mice exhibit osteopenia, feminization and metabolic syndrome. PLoS One. 2008; 3:e3858.
19. Pi M, Parrill A L, Quarles L D. GPRC6A mediates the non-genomic effects of steroids. J Biol Chem. 2010.
20. Takata R, Akamatsu S, Kubo M, Takahashi A, Hosono N, Kawaguchi T, et al. Genome-wide association study identifies five new susceptibility loci for prostate cancer in the Japanese population. Nat Genet. 2010; 42:751-4.
21. Sutters M, Yamaguchi T, Maser R L, Magenheimer B S, St John P L, Abrahamson D R, et al. Polycystin-1 transforms the cAMP growth-responsive phenotype of M-1 cells. Kidney Int. 2001; 60:484-94.
22. Mines M A, Goodwin J S, Limbird L E, Cui F F, Fan G H. Deubiquitination of CXCR4 by USP14 is critical for both CXCL12-induced CXCR4 degradation and chemotaxis but not ERK activation. J Biol Chem. 2008.
23. Varambally S, Yu J, Laxman B, Rhodes D R, Mehra R, Tomlins S A, et al. Integrative genomic and proteomic analysis of prostate cancer reveals signatures of metastatic progression. Cancer Cell. 2005; 8:393-406.
24. Beier D, Hau P, Proescholdt M, Lohmeier A, Wischhusen J, Oefner P J, et al. CD133(+) and CD133(−) glioblastoma-derived cancer stem cells show differential growth characteristics and molecular profiles. Cancer Res. 2007; 67:4010-5.
25. Gebhard C, Schwarzfischer L, Pham T H, Schilling E, Klug M, Andreesen R, et al. Genome-wide profiling of CpG methylation identifies novel targets of aberrant hypermethylation in myeloid leukemia. Cancer Res. 2006; 66:6118-28.
26. Greenberg N M, DeMayo F, Finegold M J, Medina D, Tilley W D, Aspinall J O, et al. Prostate cancer in a transgenic mouse. Proc Natl Acad Sci USA. 1995; 92:3439-43.
27. Li S, Huang S, Peng S B. Overexpression of G protein-coupled receptors in cancer cells: involvement in tumor progression. Int J. Oncol. 2005; 27:1329-39.
28. Whitehead I P, Zohn I E, Der C J. Rho GTPase-dependent transformation by G protein-coupled receptors. Oncogene. 2001; 20:1547-55.
29. Tamler R, Mechanick J I. Dietary supplements and nutraceuticals in the management of andrologic disorders. Endocrinol Metab Clin North Am. 2007; 36:533-52.
30. Sin W C, Zhang Y, Zhong W, Adhikarakunnathu S, Powers S, Hoey T, et al. G protein-coupled receptors GPR4 and TDAG8 are oncogenic and overexpressed in human cancers. Oncogene. 2004; 23:6299-303.
31. Gannon P O, Godin-Ethier J, Hassler M, Delvoye N, Aversa M, Poisson A O, et al. Androgen-regulated expression of arginase 1, arginase 2 and interleukin-8 in human prostate cancer. PLoS One. 2010; 5:e12107.
32. Delage B, Fennell D A, Nicholson L, McNeish I, Lemoine N R, Crook T, et al. Arginine deprivation and argininosuccinate synthetase expression in the treatment of cancer. Int J. Cancer. 2010; 126:2762-72.
33. Sanders J L, Chattopadhyay N, Kifor 0, Yamaguchi T, Brown E M. Ca(2+)-sensing receptor expression and PTHrP secretion in PC-3 human prostate cancer cells. Am J Physiol Endocrinol Metab. 2001; 281:E1267-74.
34. Dhanasekaran S M, Barrette T R, Ghosh D, Shah R, Varambally S, Kurachi K, et al. Delineation of prognostic biomarkers in prostate cancer. Nature. 2001; 412:822-6.
35. Liao J, Schneider A, Datta N S, McCauley L K. Extracellular calcium as a candidate mediator of prostate cancer skeletal metastasis. Cancer Res. 2006; 66:9065-73.
36. Guo C, Luttrell L M, Price D T. Mitogenic signaling in androgen sensitive and insensitive prostate cancer cell lines. J. Urol. 2000; 163:1027-32.
37. Price D T, Della Rocca G, Guo C, Ballo M S, Schwinn D A, Luttrell L M. Activation of extracellular signal-regulated kinase in human prostate cancer. J. Urol. 1999; 162:1537-42.
38. Weber M J, Gioeli D. Ras signaling in prostate cancer progression. J Cell Biochem. 2004; 91:13-25.
39. Barki-Harrington L, Daaka Y. Bradykinin induced mitogenesis of androgen independent prostate cancer cells. J. Urol. 2001; 165:2121-5.
40. Rayford W, Noble M J, Austenfeld M A, Weigel J, Mebust W K, Shah G V. Muscarinic cholinergic receptors promote growth of human prostate cancer cells. Prostate. 1997; 30:160-6.
41. Jariwala U, Prescott J, Jia L, Barski A, Pregizer S, Cogan J P, et al. Identification of novel androgen receptor target genes in prostate cancer. Mol Cancer. 2007; 6:39.
42. Yuen H F, Kwok W K, Chan K K, Chua C W, Chan Y P, Chu Y Y, et al. TWIST modulates prostate cancer cell-mediated bone cell activity and is upregulated by osteogenic induction. Carcinogenesis. 2008; 29:1509-18.
43. Papadopoulou N, Charalampopoulos I, Anagnostopoulou V, Konstantinidis G, Foller M, Gravanis A, et al. Membrane androgen receptor activation triggers down-regulation of PI-3K/Akt/NF-kappaB activity and induces apoptotic responses via Bad, FasL and caspase-3 in DU145 prostate cancer cells. Mol Cancer. 2008; 7:88.
44. Matsubara S, Wada Y, Gardner T A, Egawa M, Park M S, Hsieh C L, et al. A conditional replication-competent adenoviral vector, Ad-OC-E1a, to cotarget prostate cancer and bone stroma in an experimental model of androgen-independent prostate cancer bone metastasis. Cancer Res. 2001; 61:6012-9.
45. Bichler K H, Petri E, Hub A, Kleinknecht S, Heidenreich A. Investigations on bone metabolism of urological tumors forming metastases. Urol Int. 1996; 56:61-8.
46. Gloriam D E, Wellendorph P, Johansen L D, Thomsen A R, Phonekeo K, Pedersen D S, Brauner-Osborne H, Chemogenomic discovery of allosteric anatagonists at the GPRC6A receptor. Chem Biol. 2011 Nov. 23; 18(11):1489-98.

1. A method of characterizing a disease in a subject, comprising the steps of:
determining the level of GPRC6A in a biological sample from the subject; and
comparing the level of GPRC6A in the biological sample to a reference, wherein the disease is characterized based on a measurable difference in the level of GPRC6A in the biological sample as compared to the reference.

2. The method of claim 1, further comprising determining the amount of cyclic AMP in the biological sample from the subject and comparing the amount of cyclic AMP in the biological sample to a reference, wherein the disease is characterized based on a measurable difference in the amount of cyclic AMP in the biological sample as compared to the reference.

3. The method of claim 1, wherein the disease is prostate cancer.

4. The method of claim 3, wherein the prostate cancer is treatment-resistant prostate cancer.

5. The method of claim 1, wherein the determining and comparing said expression levels comprises isolating the GPRC6A from the biological sample of the subject.

6. The method of claim 1, wherein the determining and comparing said expression levels comprises performing an in vitro assay on the protein biomarker gene or gene products, said assay selected from the group consisting of immunoassay, histological or cytological assay, quantitative real-time PCR, and mRNA expression level assay.

7. The method of claim 1, wherein the determining and comparing said expression levels comprise
isolating the GPRC6A from the biological sample of the subject; and
performing an in vitro assay on GPRC6A, said assay selected from the group consisting of immunoassay, histological or cytological assay, quantitative real-time PCR, and mRNA expression level assay.

8. A kit for detecting prostate cancer in a subject, comprising an agent that selectively binds to a GPRC6A.

9. The kit of claim 8, wherein said agent comprises probes or primers to detect GPRC6A gene expression level.

10. The kit of claim 9, wherein the primers are selected from the group consisting of SEQ ID 1, 2, 12, 13, 14, 15, 16, 17, 18, 19.

11. The kit of claim 8, wherein the agent is an antibody.

12. A method of treating treatment-resistant prostate cancer in a subject in need thereof, comprising administering to the subject a therapeutically effective amount of an androgenergic antagonist of GPRC6A.

13. The method of claim 12, wherein the treatment-resistant prostate cancer is castration-resistant prostate cancer or a chemotherapy-resistant prostate cancer.

14. The method of claim 12, wherein the androgenergic antagonist is selected from the group consisting of allylestrenol, oxendolone, osaterone acetate, bicalutamide, steroidal anti-androgergic agents, medroxyprogesterone (MPA), cyproterone, cyproterone acetate (CPA), dienogest, flutamide, nilutamide, spironolactone, 5alpha-reductase inhibitors, dutasteride, finasteride, salts thereof, gold nanoparticles thereof, combinations thereof, and the like.

15. The method of claim 12 wherein the androgenergic antagonist is a gold nanoparticle of α-bicalutamide.

16. The method of claim 12, wherein the androgenergic antagonist is a gold nanoparticle of β-bicalutamide.

17. The method of claim 12, wherein the likelihood of a positive therapeutic effect of said androgenergic antagonist can be predicted by determining the amount of cyclic AMP in a biological sample from the subject before and after administration of said androgenergic antagonist.

18. The method of claim 16, wherein the androgenergic antagonist is bicalutamide.

19. The method of claim 16, wherein the androgenergic antagonist is a gold nanoparticle α-bicalutamide.

20. The method of claim 16, wherein the androgenergic antagonist is a gold nanoparticle of β-bicalutamide.


SEQUENCE LISTING (19 sequences)

SEQ ID NO: 1 - DNA, 20 bp, Artificial Sequence
  Other information Synthetic
caggagtgtgttggctttga

SEQ ID NO: 2 - DNA, 20 bp, Artificial Sequence
  Other information Synthetic
atcaggtgagccattgcttt

SEQ ID NO: 3 - DNA, 25 bp, Artificial Sequence
  Other information Synthetic
gaccccttcattgacctcaactaca

SEQ ID NO: 4 - DNA, 24 bp, Artificial Sequence
  Other Information Synthetic
ggtcttactccttggaggccatgt

SEQ ID NO: 5 - DNA, 20 bp, Artificial sequence
  Other Information Synthetic
ttggaaatgaccaggccaag

SEQ ID NO: 6 - DNA, 20 bp, Artificial sequence
  Other Information Synthetic
agcaaccctggacctcacac

SEQ ID NO: 7 - DNA, 21 bp, Artificial sequence
  Other Information Synthetic
attcctgtagatccgagcacc

SEQ ID NO: 8 - DNA, 20 bp, Artificial sequence
  Other Information Synthetic
gctcacgtcgctcattttgc

SEQ ID NO: 9 - DNA, 2860 bp, Homo sapiens
actgagcaaatgagatagaaacatggcattcttaattatactaattacctgctttgtgattattcttgctacttcacagccttgccagacccctgatgactttgtggctgccacttctccgggacatatcataattggaggtttgtttgctattcatgaaaaaatgttgtcctcagaagactctcccagacgaccacaaatccaggagtgtgttggctttgaaatatcagtttttcttcaaactcttgccatgatacacagcattgagatgatcaacaattcaacactcttacctggagtcaaactggggtatgaaatctatgacacttgtacagaagtcacagtggcaatggcagccactctgaggtttctttctaaattcaactgctccagagaaactgtggagtttaagtgtgactattccagctacatgccaagagttaaggctgtcataggttctgggtactcagaaataactatggctgtctccaggatgttgaatttacagctcatgccacaggtgggttatgaatcaactgcagaaatcctgagtgacaaaattcgctttccttcatttttacggactgtgcccagtgacttccatcaaattaaagcaatggctcacctgattcagaaatctggttggaactggattggcatcataaccacagatgatgactatggacgattggctcttaacacttttataattcaggctgaagcaaataacgtgtgcatagccttcaaagaggttcttccagcctttctttcagataataccattgaagtcagaatcaatcggacactgaagaaaatcattttagaagcccaggttaatgtcattgtggtatttctgaggcaattccatgtttttgatctcttcaataaagccattgaaatgaatataaataagatgtggattgctagtgataattggtcaactgccaccaagattaccaccattcctaatgttaaaaagattggcaaagttgtagggtttgcctttagaagagggaatatatcctctttccattcctttcttcaaaatctgcacttgcttcccagtgacagtcacaaactcttacatgaatatgccatgcatttatctgcctgcgcatatgtcaaggacactgatttgagtcaatgcatattcaatcattctcaaaggactttggcctacaaggctaacaaggctatagaaaggaacttcgtcatgagaaatgacttcctctgggactatgctgagccaggactcattcatagtattcagcttgcagtgtttgcccttggttatgccattcgggatctgtgtcaagctcgtgactgtcagaaccccaacgcctttcaaccatgggagttacttggtgtgctaaaaaatgtgacattcactgatggatggaattcatttcattttgatgctcacggggatttaaatactggatatgatgttgtgctctggaaggagatcaatggacacatgactgtcactaagatggcagaatatgacctacagaatgatgtcttcatcatcccagatcaggaaacaaaaaatgagttcaggaatcttaagcaaattcaatctaaatgctccaaggaatgcagtcctgggcaaatgaagaaaactacaagaagtcaacacatctgttgctatgaatgtcagaactgtcctgaaaatcattacactaatcagacagatatgcctcactgccttttatgcaacaacaaaactcactgggcccctgttaggagcactatgtgctttgaaaaggaagtggaatatctcaactggaatgactccttggccatcctactcctgattctctccctactgggaatcatatttgttctggttgttggcataatatttacaagaaacctgaacacacctgttgtgaaatcatccgggggattaagagtctgctatgtgatccttctctgtcatttcctcaattttgccagcacgagctttttcattggagaaccacaagacttcacatgtaaaaccaggcagacaatgtttggagtgagctttactctttgcatctcctgcattttgacgaagtctctgaaaattttgctagccttcagctttgatcccaaattacagaaatttctgaagtgcctctatagaccgatccttattatcttcacttgcacgggcatccaggttgtcatttgcacactctggctaatctttgcagcacctactgtagaggtgaatgtctccttgcccagagtcatcatcctggagtgtgaggagggatccatacttgcatttggcaccatgctgggctacattgccatcctggccttcatttgcttcatatttgctttcaaaggcaaatatgagaattacaatgaagccaaattcattacatttggcatgctcatttacttcatagcttggatcacattcatccctatctatgctaccacatttggcaaatatgtaccagctgtggagattattgtcatattaatatctaactatggaatcctgtattgcacattcatccccaaatgctatgttattatttgtaagcaagagattaacacaaagtctgcctttctcaagatgatctacagttattcttcccatagtgtgagcagcattgccctgagtcctgcttcactggactccatgagcggcaatgtcacaatgaccaatcccagctctagtggcaagtctgcaacctggcagaaaagcaaagatcttcaggcacaagcatttgcacacatatgcagggaaaatgccacaagtgtatctaaaactttgcctcgaaaaagaatgtcaagtatatgaataagccttaggagatgccacattccagaataaaatgtttccagggtctttgcatct

SEQ ID NO: 10 - DNA, 19 bp, Artificial sequence
  Other information Synthetic
ccaggagtgtgttggcttt

SEQ ID NO: 11 - DNA, 19 bp, Artificial sequence
  Other Information Synthetic
gccacaggtgggttatgaa

SEQ ID NO: 12 - DNA, 31 bp, Artificial sequence
  Other Information synthetic
gaataactagcaggaggggcgctggaaggag

SEQ ID NO: 13 - DNA, 31 bp, Artificial sequence
  Other Information Synthetic
cagagtggcagccattgctgctgtgacttcg

SEQ ID NO: 14 - DNA, 28 bp, Artificial sequence
  Other Information Synthetic
cacgagagatcgtggggtatcgacagag

SEQ ID NO: 15 - DNA, 33 bp, Artificial sequence
  Other Information Synthetic
ctacatggcgtgatttcatatgcgcgattgctg

SEQ ID NO: 16 - DNA, 20 bp, Artificial sequence
  Other Information Synthetic
caaatgttgcttgtctggtg

SEQ ID NO: 17 - DNA, 20 bp, Artificial sequence
  Other information Synthetic
gtcagtcgagtgcacagttt

SEQ ID NO: 18 - DNA, 21 bp, Artificial sequence
  Other Information Synthetic
cagagcagaattgtggagtgg

SEQ ID NO: 19 - DNA, 26 bp, Artificial Sequence
  Other information Synthetic
ggacaaaccacaactagaatgcagtg